(19) United States
(12) Patent Application Publication
(10) Pub. No.: US 2024/0108798 A1
(43) Pub. Date: Apr. 4, 2024

(54) CLEANING DEVICE AND METHOD FOR NASAL CAVITIES

(71) Applicant: Kemi L. Azeez, Lima, OH (US)

(72) Inventors: Kemi L. Azeez, Lima, OH (US); David Michael Uhlenhake, Toledo, OH (US); Pratik Roopchand Baldota, Mumbai (IN)

(21) Appl. No.: 18/477,669

(22) Filed: Sep. 29, 2023

Related U.S. Application Data
(60) Provisional application No. 63/411,420, filed on Sep. 29, 2022.

(51) Int. Cl. A61M 3/02

(52) U.S. Cl. CPC A61M 3/0258

(57) ABSTRACT

Embodiments of the present disclosure may include a chargeable nasal cleaning device including a housing, head assembly, and fluid tank. The head assembly may include a rotatable tip and a basin circumscribing the rotatable tip. The rotatable tip may oscillate +/−30 degrees and may be replaceable. The basin may catch solid waste released from a nasal cavity and further drain the liquid waste. The fluid tank may include a clean reservoir with a liquid saline solution and a waste reservoir provided to collect the liquid waste released from the nasal cavity. The clean reservoir may be in fluid communication with the rotatable tip of the head assembly and the waste reservoir may be in fluid communication with the basin of the head assembly. A method of using a nasal cleaning device is further provided.

### CROSS-REFERENCE TO RELATED APPLICATIONS

This application claims the benefit of U.S. Provisional Application No. 63/411,420, filed on Sep. 29, 2022. The entire disclosure of the above application is incorporated herein by reference.

### FIELD

The present technology relates to cleaning nasal cavities, including a cleaning device and method for utilizing a nasal cleaning device.

### INTRODUCTION

This section provides background information related to the present disclosure which is not necessarily prior art.

Throughout the course of a day, the nasal cavity may accumulate mucus, dust, bacteria, viruses, and fungi. The presence of such foreign substances can trigger problems, such as infections, diseases, and sicknesses. The nose acts as a first line of defense against these foreign substances. According to experts, a clear nasal passage may help protect the body and help achieve a well-rested sleep. Proper nasal health may be important for conditioning the air that is breathed and for maintaining a disease-free body, decreasing allergy effects, and improving chronic allergy symptoms.

Cleaning the nasal cavity may help rid the nasal passages and sinuses of contaminants. Cleaning the nasal cavity may also increase moisture of the nasal mucosa and the membrane of the nose cavity, improve breathing, reduce nose blockage, decrease nose sensitivity to dust, and nasal inflammation, help wash out thick mucus, including stuffy nose, itchiness, runny nose, and pain. Current methods of cleaning the nasal cavity may include scraping, sponging, and flushing the cavity with a saline solution or other sterile solution. Such methods, however, may provide an incomplete cleaning. Saline or other sterile solutions may also cause discomfort to a user as the fluid is flushed through each side of the nasal cavity. Moreover, devices for cleaning the nasal cavity may be hard to clean themselves, complicated to use, and expensive.

Accordingly, there is a need for a nasal cleaning device, which is easy to clean, which is easy use, and which provides a complete and thorough cleaning of the nasal cavity.

### SUMMARY

In concordance with the instant disclosure, a nasal cleaning device, which is easy to clean, which is easy use, and which provides a complete and thorough cleaning of the nasal cavity, has surprisingly been discovered.

In certain embodiments, a nasal cleaning device is provided. The nasal cleaning device may include a housing including a first end and a second end, a head assembly including a rotatable tip and a basin circumscribing the rotatable tip, and a fluid tank disposed on the second end of the housing wherein the fluid tank may include a clean reservoir and a waste reservoir. The clean reservoir may be in fluid communication with the rotatable tip of the head assembly via a first tube, a second tube, a third tube, and a diaphragm pump. The waste reservoir may be in fluid communication with the basin of the head assembly via a fourth tube configured to permit a gravity flow from the basin of the head assembly to the waste reservoir. The rotatable tip may be configured to oscillate +/−30 degrees about a vertical axis and may be replaceable for each use.

In another embodiment, a method of using the nasal cleaning device is provided. The method including steps of providing a nasal cleaning device and inserting the rotatable tip into a nasal cavity. The steps further include turning the nasal cleaning device on and transferring a solution from the clean reservoir of the fluid tank to the nasal cavity. The steps may also include collecting fluid in the basin of the head assembly and transferring the fluid to the waste reservoir of the fluid tank. The method may further include steps of turning the nasal cleaning device off and removing the rotatable tip from the nasal cavity.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a top perspective view of a nasal cleaning device according to an embodiment of the present disclosure;

FIG. 2 is a bottom perspective view of the nasal cleaning device shown in FIG. 1;

FIG. 3 is a top plan view of the nasal cleaning device shown in FIG. 1;

FIG. 4 is a front elevational view of the nasal cleaning device shown in FIG. 1;

FIG. 5 is a bottom plan view of the nasal cleaning device shown in FIG. 1;

FIG. 6 is a rear elevational view of the nasal cleaning device shown in FIG. 1;

FIG. 7 is a left-side elevational view of the nasal cleaning device shown in FIG. 1;

FIG. 8 is a right-side elevational view of the nasal cleaning device shown in FIG. 1;

FIG. 9 is an exploded view of the nasal cleaning device shown in FIG. 1;

FIG. 10 is a cross-sectional view thereof taken along section line 10-10 in FIG. 3;

FIG. 11 is a cross-sectional view thereof taken along section 11-11 in FIG. 7;

FIG. 12 is a top perspective view of the nasal cleaning device shown in FIG. 1 with a cap removed therefrom;

FIG. 13 is a front elevational view of the nasal cleaning device without the removable cap shown in FIG. 12;

FIG. 14 is a top plan view of the nasal cleaning device shown in FIG. 12;

FIG. 15 is a portion of the head assembly without the removable cap shown in FIG. 12;

FIG. 16 is a cross-sectional view of the head assembly without the removable cap shown in FIG. 15;

FIG. 17 depicts the fluid tank from the call-out shown in FIG. 11;

FIG. 18 is a portion of the internal components of the nasal cleaning device;

FIG. 19 is a portion of the gear assembly shown in FIG. 18;

FIG. 20 is an environmental view of the nasal cleaning device inserted in a nasal cavity; and

FIG. 21 is a flowchart depicting a method of using the nasal cleaning device, according to an embodiment of the present disclosure.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps may be different in various embodiments, including where certain steps may be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9,1-8,1-3,1-2,2-10,2-8,2-3,3-10,3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” may encompass both an orientation of above and below. The device may be otherwise oriented (rotated 90 degrees or at other orientations) and the spatially relative descriptors used herein interpreted accordingly.

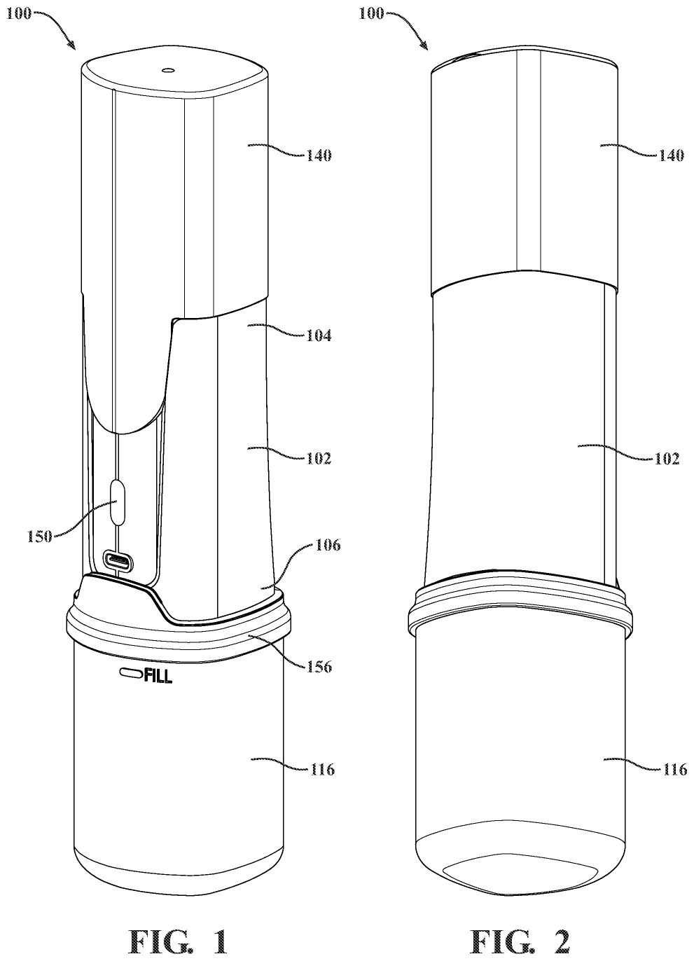

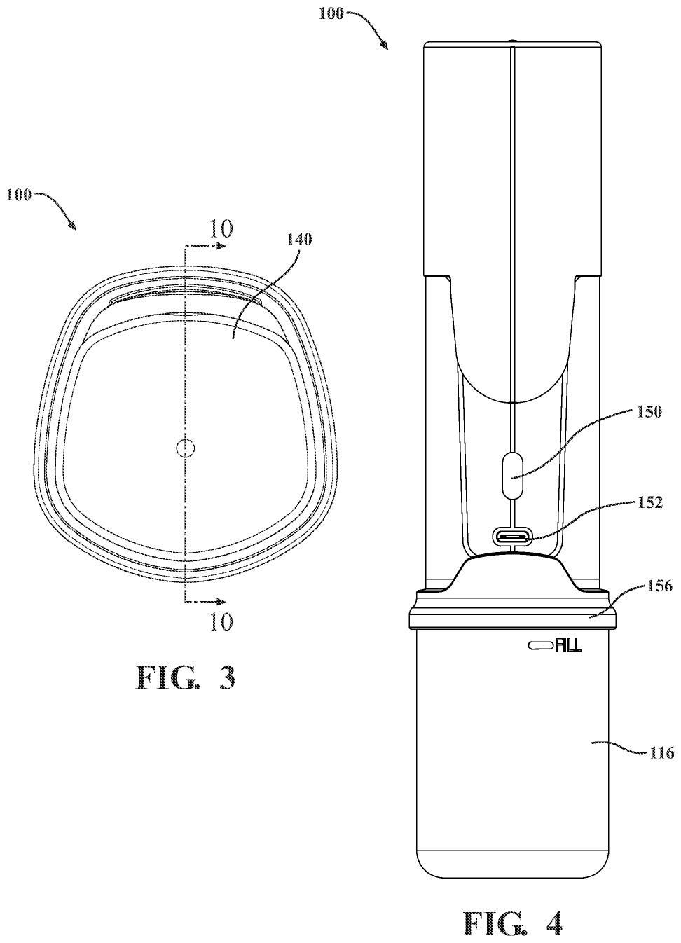

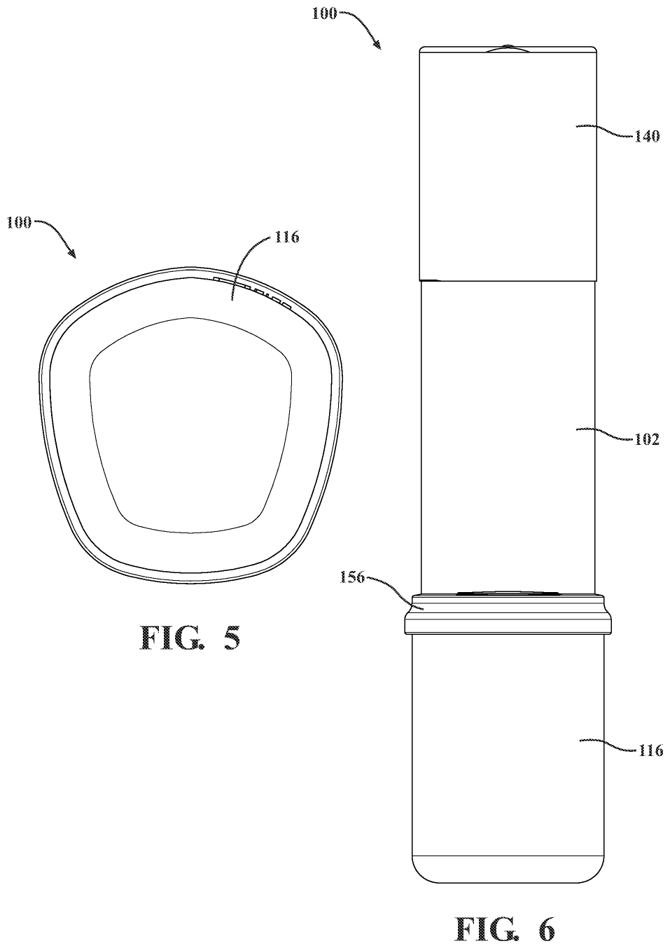

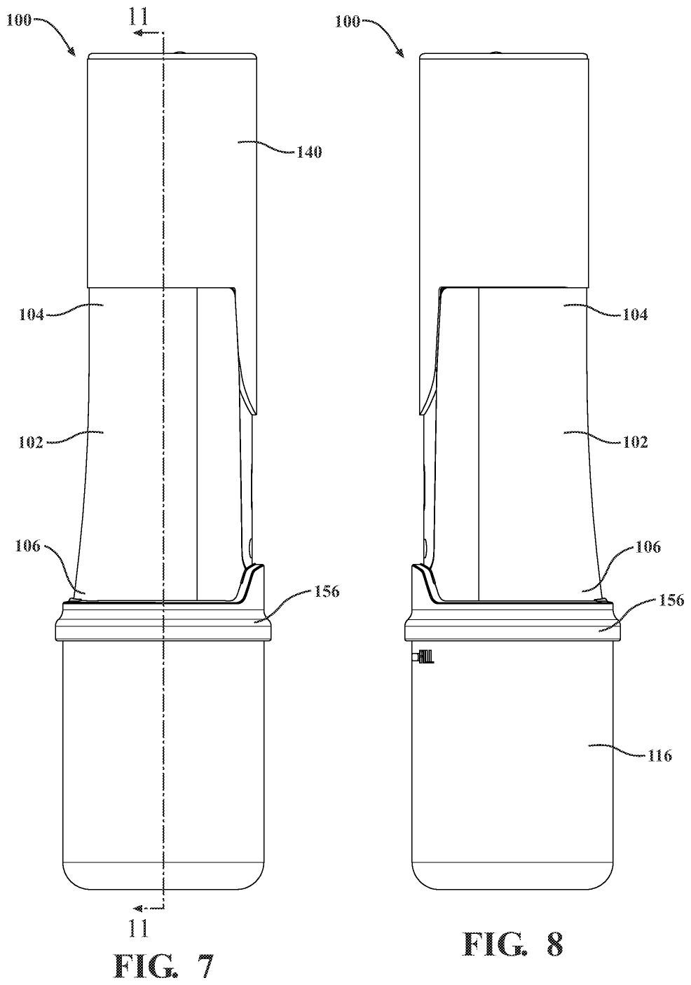

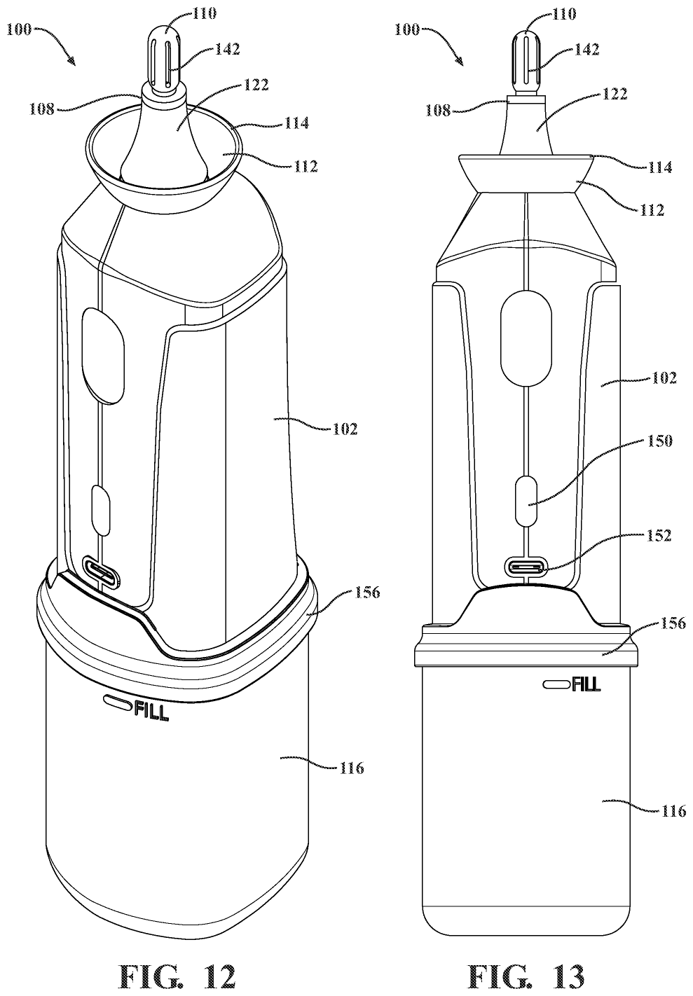

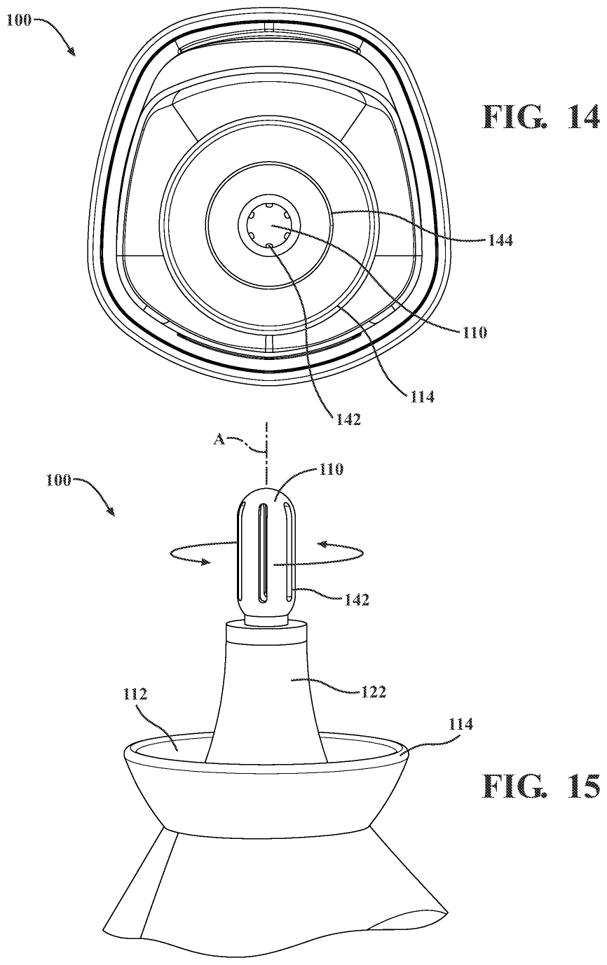

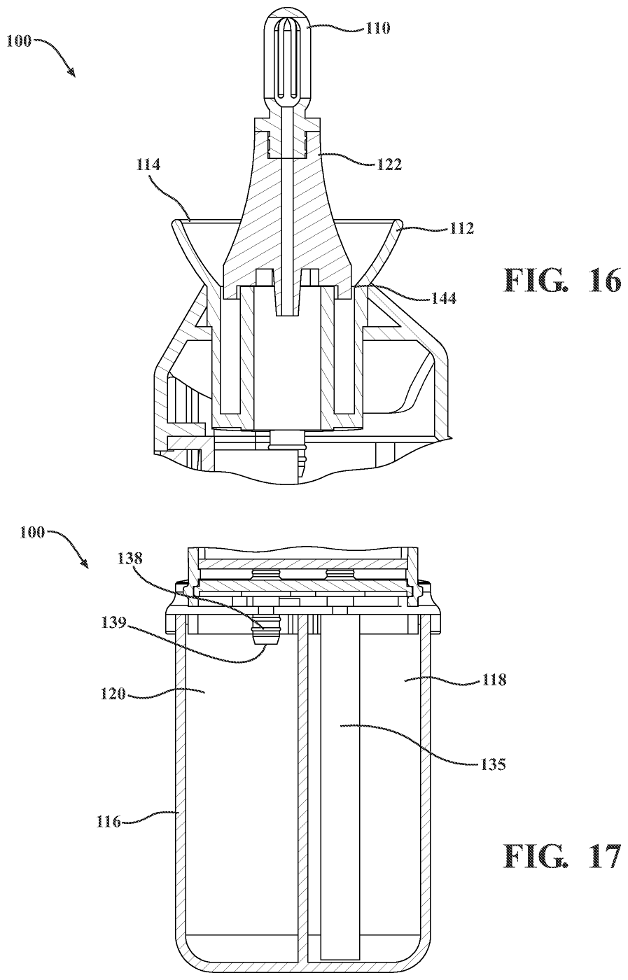

The present technology relates to a nasal cleaning device 100, shown generally in accompanying FIGS. 1-21. Advantageously, the nasal cleaning device 100 provides an easy and efficient device to clean a nasal cavity 101 with a rotatable tip 110 that is easily removable and replaceable. Ways of using the nasal cleaning device 100 are also provided.

An embodiment of the nasal cleaning device 100 in accordance with the present disclosure is shown in FIGS. 1-20. Generally, the nasal cleaning device 100 may include a housing 102, a head assembly 108 including a rotatable tip 110 and a basin 112, as well as a fluid tank 116 including a clean reservoir 118 and a waste reservoir 120.

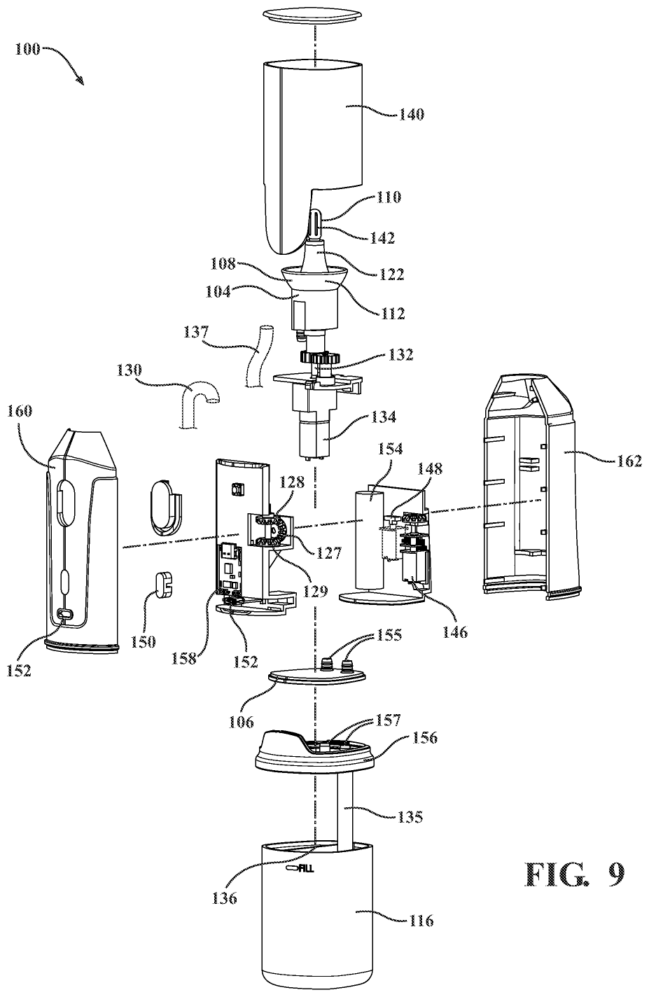

The housing 102 may include the following aspects. The housing 102 may include a first end 104 and a second end 106, as shown in FIGS. 1-2, 4, 6, and 7-8. The head assembly 108 may be disposed on the first end 104 of the housing 102 and the fluid tank 116 may be removably disposed on the second end 106 of the housing 102. With reference to FIGS. 9-11 and 18, the housing 102 may also include a drive shaft 124 which may further include a gear assembly 126 including a first gear 127, a second gear 128, and a third gear 129 in communication with the rotatable tip 110 of the head assembly 108, as further described herein.

With reference to FIGS. 9, 11, and 18, the housing 102 may also include a first motor 146 and a second motor 148. The first motor 146 may be coupled to the gear assembly 126 and be in communication with the rotatable tip 110 of the head assembly 108 in order to drive the rotation of the rotatable tip 110. The second motor 148 may be coupled to a diaphragm pump 134 in order to provide fluid communication between the clean reservoir 118 and the rotatable tip 110.

With reference to FIGS. 1, 4, 9, and 18, the housing 102 may also include an electrical system 158 disposed within the housing 102. The electrical system 158 may include a rechargeable battery 154, a charging port 152, a power button 150, an indicator, a controller, and user interface. The rechargeable battery 154 may be a replaceable lithium battery. One of ordinary skill in the art may select suitable rechargeable batteries 154. The charging port 152 may be a USB-C port and may further be disposed on an outside surface of the housing 102 near the power button 150. The electrical system 158 may also include a power button 150 disposed on the outside surface of the housing 102. The power button 150 may work in conjunction with the electrical system 158 such that pushing the power button 150 turns the nasal cleaning device 100 on to power the rotation of the rotatable tip 110. Pressing the power button 150 a second time may also power the nasal cleaning device 100 off. In another embodiment, holding down the power button 150 may power the nasal cleaning device 100 on to power the rotation of the rotatable tip 110 and releasing the power button 150 may turn off the nasal cleaning device 100. One of ordinary skill in the art may select suitable arrangements to power the device within the scope of the present disclosure. The indicator may be located above the power button 150 on the outside surface of the housing 102. Advantageously, the indicator may light up when the power button 150 is pressed and the nasal cleaning device 100 is turned on and may also light up when the nasal cleaning device 100 is in in a charging site.

With reference to FIG. 9, the housing 102 may be comprised of a first housing wall 160 and a second housing wall 162 that are coupled together to define the housing 102 and designed to hold the internal components of the nasal cleaning device 100 in place. An interior housing wall may be disposed inside the housing 102 and may further separate the internal components to protect the electrical system 158. The internal components may be fastened together using various fasteners. As a non-limiting example, O-ring fasteners may be used. The internal components may also be press-fit and connected via an adhesive. As a non-limiting example, a secondary mold operation may be utilized to apply a flexible edge for sealing the components or a silicone adhesive may be used to seal the components.

With reference to FIGS. 9-16, the rotatable tip 110 of the head assembly 108 may include the following aspects. The rotatable tip 110 of the head assembly 108 may be configured to rotate through at least an entire revolution. The rotatable tip 110 may also be configured to oscillate. The rotatable tip 110 may oscillate +/−30 degrees about a vertical axis (A), as shown in FIG. 15. In another embodiment, the rotatable tip 110 may oscillate at various degrees relative to the vertical axis (A). Alternatively, as a non-limiting example, the rotatable tip 110 may oscillate +/−60 degrees relative to the vertical axis (A). One of ordinary skill in the art may select suitable configurations and degrees to oscillate the rotatable tip 110 relative to the vertical axis (A) within the scope of the present disclosure. Advantageously, this oscillation is designed to provide a thorough cleaning of a nasal cavity 101. The rotatable tip 110 may rotate or oscillate in a nasal cavity 101 to dislodge bacteria and allergens from the nasal cavity 101. One of ordinary skill in the art may select alternate rotational directions to power the rotatable tip 110 within the scope of the present disclosure. The rotatable tip 110 of the head assembly 108 may be removably attached to the head assembly 108. Advantageously, the rotatable tip 110 may be easily removed and replaced to continuously provide a clean tip for each use and to switch out the rotatable tip 110 of various sizes. The rotatable tip 110 may be disposed entirely above a lip 114 of the basin 112, as shown in FIGS. 9, 12-13, and 15-16. The rotatable tip 110 may include a plurality of channels 142 in fluid communication with the clean reservoir 118. The plurality of channels 142 may be disposed anywhere from a selected portion to an entirety of the rotatable tip 110 to provide fluid flow to the nasal cavity 101. Alternatively, the rotatable tip 110 may include a single aperture or a plurality of apertures in fluid communication with the clean reservoir 118.

A solution used for cleaning the nasal cavity is fluidly coupled between the clean reservoir 118 and the plurality of channels 142 of the rotatable tip 110. Examples of the solution include a saline solution, water, and other sterile solutions suitable for cleaning the nasal cavity 101. Streams of the solution may be directed out of the plurality of channels 142 or apertures as the solution is received from the clean reservoir 118. The solution may be sprayed or misted directly out of the plurality of channels 142 or apertures. More specifically, when the nasal cleaning device 100 is turned on by engaging the power button 150, the rotatable tip 110 receives the solution from the clean reservoir 118 whereby the solution sprays out of the plurality of channels 142 of the rotatable tip 110. The power button 150 can therefore actuate the first motor 146 to drive rotation of the rotatable tip 110 and actuate the second motor 148 to drive the diaphragm pump 134 to deliver the liquid solution from the clean reservoir 118 to the rotatable tip 110.

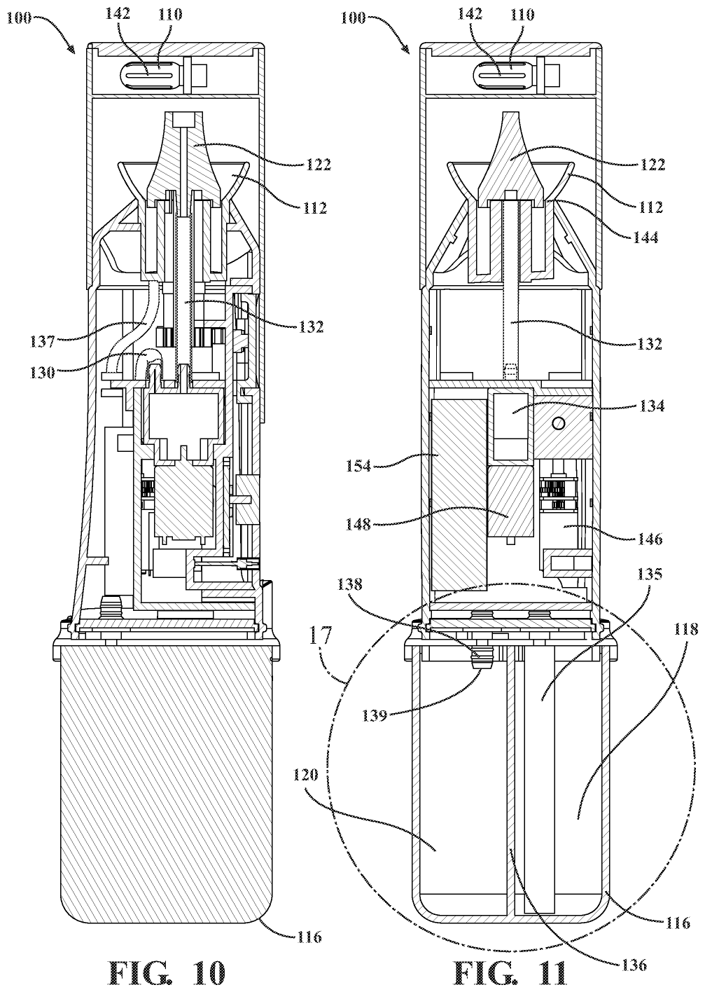

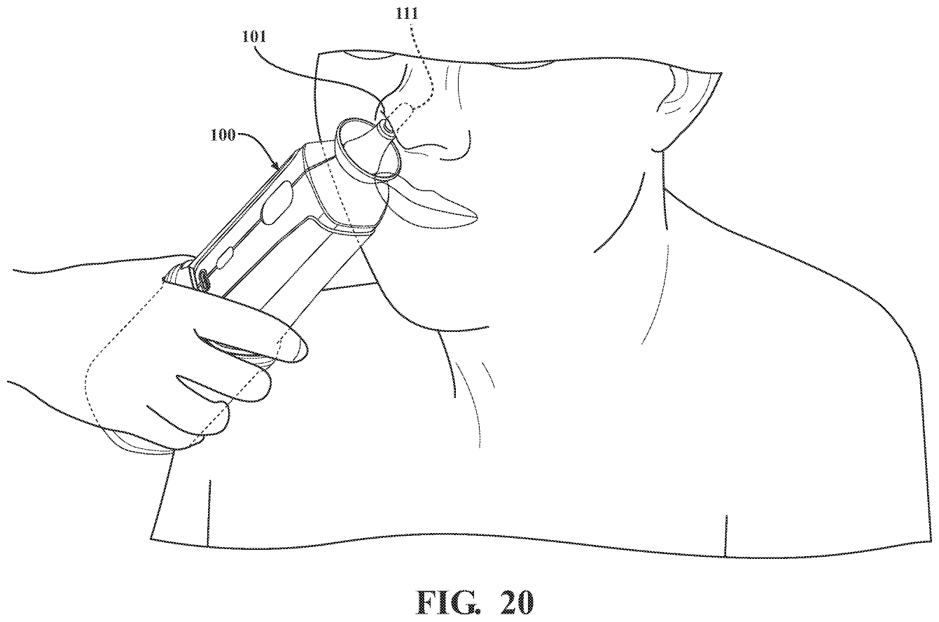

In certain embodiments, a soft material 111 may cover anywhere from a selected portion to an entirety of the rotatable tip 110 to provide comfort to the user when the nasal cleaning device 100 is in use and inserted into a nasal cavity 101, as shown in FIG. 20. This soft material 111 may include cotton. One of ordinary skill in the art may select suitable materials 111 to cover the rotatable tip 110. The soft material 111 and/or the rotatable tip 110 itself may be replaceable. A removable cap 140 for the nasal cleaning device 100 can be sized to accommodate the rotatable tip 110 in an installed position (e.g., FIGS. 9, 12-13, and 15-16) or in an uninstalled position (e.g., FIGS. 10-11). It is possible to have the removable cap 140 include a compartment or cavity to store the rotatable tip 110, as well as one or more extra rotatable tips 110. Advantageously, the compartment or cavity of the removable cap 140 may store an extra rotatable tip 110 to militate against misplacement of the rotatable tip 110 when in the uninstalled position.

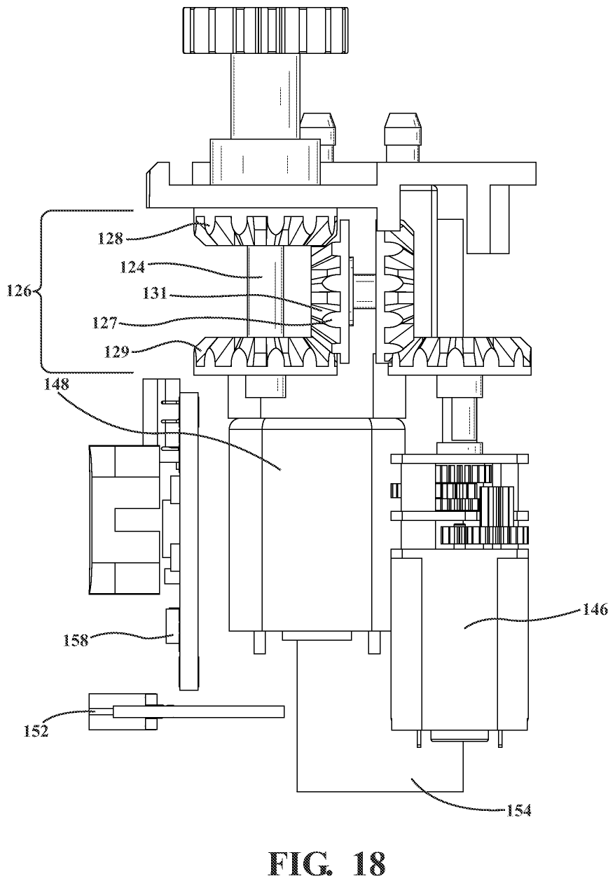

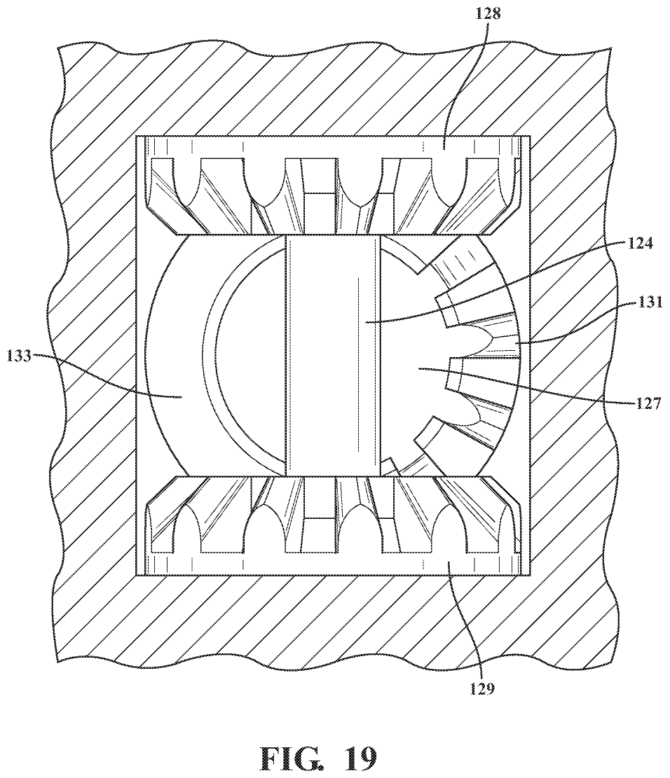

As described herein, the first motor 146 may be in communication with the gear assembly 126 to drive the rotation of the rotatable tip 110 while the second motor 148 may be in communication with the diaphragm pump 134 to deliver the liquid solution from the clean reservoir 118 to the rotatable tip 110. Specifically, the first motor 146 may drive the rotation of the rotatable tip 110. With reference to FIGS. 18 and 19, a first gear 127 may be coupled to the first motor 146 while a second gear 128 and a third gear 129 may be directly coupled to the drive shaft 124. The drive shaft 124 may be coupled to the rotatable tip 110 of the head assembly 108. The first gear 127 may have an engagement portion 131 and a nonengagement portion 133 such that rotation of the first gear 127 may result in the engagement portion 131 engaging one of the second gear 128 and the third gear 129 and the nonengagement portion 133 engaging the other of the second gear 128 and the third gear 129. Further, engagement of the second gear 128 may cause the second gear 128 to rotate in a first direction and engagement of the third gear 129 may cause the third gear 129 to rotate in a second direction. The first direction may be clockwise and the second direction may be counterclockwise relative to the vertical axis (A), or vice versa. In this way, revolutions of the first gear 127 result in the engagement portion 131 alternatively engaging one of the second gear 128 and the third gear 129, causing the drive shaft 124 to rotate in alternate directions; e.g., oscillate.

In another embodiment, the rotatable tip 110 may also pivot relative to the vertical axis (A), shown in FIG. 15. In a further embodiment, the rotatable tip 110 may move axially along the vertical axis (A), allowing the rotatable tip 110 to extend or retract by moving up and down. Generally, the direction of rotation of the rotatable tip 110 may be caused by the configuration of the gear assembly 126. One of ordinary skill in the art may select suitable configurations to drive the rotation of the rotatable tip 110 within the scope of the present disclosure.

The head assembly 108 may also include a rotatable tip driver 122 connected to the rotatable tip 110, as shown in FIGS. 9-13, and 15-16. The rotatable tip driver 122 may be removably attached to the head assembly 108. Both the rotatable tip driver 122 and the rotatable tip 110 may be removably attached to the head assembly 108 for convenience and for easily replacing the rotatable tip 110. The rotatable tip driver 122 may be disposed substantially through the basin 112 with a portion disposed above the lip 114 of the basin 112. The rotatable tip driver 122 holds the rotatable tip 110 above the lip 114 of the basin 112 and may further drive the rotation of the rotatable tip 110 when the nasal cleaning device 100 is powered on.

The basin 112 of the head assembly 108 may include the following aspects. The basin 112 may circumscribe the rotatable tip 110 of the head assembly 108, as shown in FIGS. 9-16. The basin 112 may also circumscribe the rotatable tip driver 122. The basin 112 is configured to catch waste, including solid and/or liquid waste, released from the nasal cavity 101 when the nasal cleaning device 100 is in use. The basin 112 may also include an aperture 144 disposed within the basin 112. More specifically, the waste reservoir 120 may be in fluid communication with the basin via the aperture 144. The aperture 144 may be designed to drain liquid waste released from the nasal cavity 101 into the basin 112 while the basin 112 holds any solid waste released from the nasal cavity 101. The aperture 144 may be narrow in order to militate against any solid waste from being drained from the basin 112. As further described herein, the size of the aperture 144 may depend on the size and diameter of the fourth tube 137. In another embodiment, the basin 112 may have one or more apertures 144 to drain the liquid waste released from the nasal cavity 101. One of ordinary skill in the art may select suitable shapes and apertures to dispose within the basin 112, within the scope of the present disclosure.

The fluid tank 116 may include the following aspects. The fluid tank 116 may be in communication with the head assembly 108. The fluid tank 116 may be removably disposed on the second end 106 of the housing 102. The fluid tank 116 may be coupled to the second end 106 of the housing via a removable cuff 156, as shown in FIGS. 1-2, 4, 6, 7-13, and 17. Advantageously, the fluid tank 116 and removable cuff 156 may be taken off the second end 106 of the housing 102 to allow the nasal cleaning device 100 to rest flat on a surface when not in use or when stored away. Alternatively, the fluid tank 116 alone may be removed from the second end 106 of the housing 102 without taking off the removable cuff 156. The fluid tank 116 may be transparent to provide a visual indication of fluid within the fluid tank 116. Advantageously, the transparency may indicate that more fluid should be added to the fluid tank 116 or that the liquid waste in the waste reservoir 120 must be drained or removed. Desirably, the fluid tank 116 contains a fill line that is visible to the user to indicate to the user how much to fill the fluid tank 116 without any overflow.

With continued reference to the fluid tank 116, shown generally in FIGS. 1-18, the fluid tank 116 may include a clean reservoir 118 and a waste reservoir 120 whereby the clean reservoir 118 may contain a solution. The solution in the clean reservoir 118 may be a liquid saline solution or water. One of ordinary skill in the art may select a suitable liquid solution to fill the clean reservoir 118 for cleaning a nasal cavity 101. As a non-limiting example, the clean reservoir 118 may contain a volume of 2 ounces of the solution. One of ordinary skill in the art may select a suitable size and volume of the clean reservoir 118 within the scope of the present disclosure.

The clean reservoir 118 of the fluid tank 116 may be in fluid communication with the rotatable tip 110 of the head assembly 108. More specifically, the clean reservoir 118 may be in fluid communication with the rotatable tip 110 of the head assembly 108 via a first tube 130, a second tube 132, a third tube 135, and the diaphragm pump 134, all in fluid communication with each other. The first tube 130 may be in fluid communication with the second tube 132 and the third tube 135. The third tube 135 may extend into the clean reservoir 118 and be disposed inside the clean reservoir 118, as shown in FIGS. 11 and 17. The first tube 130 may connect the third tube 135 disposed inside the clean reservoir 118 to the diaphragm pump 134 to deliver the solution from the clean reservoir 118 to the diaphragm pump 134. The second tube 132 may connect the diaphragm pump 134 to the rotatable tip 110 to deliver the solution to the rotatable tip 110, as shown in FIG. 10. Advantageously, the solution from the clean reservoir 118 travels from the clean reservoir 118 of the fluid tank 116 to the rotatable tip 110 through the first tube 130 and the second tube 132, thereby protecting the internal components and electrical system 158 held in the housing 102. The diaphragm pump 134 may be configured to provide fluid flow from the clean reservoir 118 to the rotatable tip 110 of the head assembly 108 through the first tube 130, the second tube 132, and the third tube 135. The diaphragm pump 134 may be coupled to the second motor 148 to power the diaphragm pump 134. When the power button 150 is engaged, the first motor 146 and the second motor 148 may activate whereby the first motor 146 may power the gear assembly 126 to drive the rotation of the rotatable tip 110 while the second motor 148 may simultaneously power the diaphragm pump 134 to provide fluid flow to the rotatable tip 110. In another embodiment, a second button (not pictured) may be disposed adjacent the power button 150 whereby the second button may power the diaphragm pump 134 to provide fluid flow to the rotatable tip 110 while the power button 150 may activate the rotation of the rotatable tip 110, or vice versa. In another embodiment, the clean reservoir 118 may include a filter coupled to the third tube 135 to militate against potential blockage of the rotatable tip 110 from solid particles.

Additionally, the waste reservoir 120 may contain the liquid waste released from the nasal cavity 101. The waste reservoir 120 may be in fluid communication with the basin 112 of the head assembly 108. In particular, the waste reservoir 120 may be in fluid communication with the basin 112 of the head assembly 108 via a fourth tube 137. The fourth tube 137 may connect the aperture 144 of the basin 112 to the waste reservoir 120. The fourth tube 137 may be configured to permit a gravity flow from the aperture 144 of the basin 112, which thereby collects the liquid waste released from the nasal cavity 101 into the basin 112, through the aperture 144, and into the waste reservoir 120. As a non-limiting example, the diameter of the aperture 144 of the basin 112 may depend on the diameter of the fourth tube 137. More specifically, the diameter of the tubing may be calculated as the cross-sectional diameter of a circular cross-sectioned tubing and the diameter of the aperture 144 may be calculated as the distance of empty space circumscribing the basin 112 from the rotatable tip driver 122. For example, a 5-millimeter tube may be coupled to a 1.25-millimeter aperture. Alternatively, as another example, a 3-millimeter tube may be coupled to a 1-millimeter aperture. The diameter of the aperture 144 may depend on the diameter of the tubing for the fourth tube 137 to militate against blockage. As a non-limiting example, the diameter of the aperture 144 may range from 0.5 millimeters to 2 millimeters. One of ordinary skill in the art may select suitable diameters for the fourth tube 137 and the aperture 144 within the scope of the present disclosure. Alternatively, in another embodiment, a filter may be coupled to the aperture 144 of the basin 112 to collect solid waste released from the nasal cavity 101.

The fourth tube 137 need not extend through the waste reservoir 120 due to the gravity flow mechanism which allows the liquid waste to simply travel through the fourth tube 137 as it connects from the aperture 144 of the basin 112 all the way to a drain 138 having a drainage aperture 139 formed therethrough. The drain 138 may be disposed adjacent the waste reservoir 120 and may be coupled to the fourth tube 137. Advantageously, the drain 138 may be configured to drain potential leakage into the waste reservoir 120 and may further be coupled to the fourth tube 137 to bring any and all liquid waste down by a gravity flow to the waste reservoir 120. This militates against any potential exposure or mixture of the liquid waste with the clean reservoir 118.

In particular embodiments, for example, as shown in FIG. 9, it should be understood that the first tube 130 and the fourth tube 137 may be coupled to and in fluid communication with raised studs 155 formed on an inner surface of the second end 106 of the housing 102. Likewise, the raised studs 155 are coupled to and placed in fluid communication with corresponding studs 157 formed on an outer surface of the removable cuff 156 when the device is fully assembled for use. This permits for fluid flow both away from the clean water reservoir, and returning back to the dirty water reservoir, in a manner that also facilitates the removability of the reservoirs from the device for refilling or cleaning, for example. Other suitable means for placing the first tube 130 and the fourth tube 137 in fluid communication with the reservoirs may also be employed within the scope of the present disclosure, as desired.

Certain embodiments of the nasal cleaning device 100 can include the following aspects. The fluid tank 116 may include a wall 136 to separate the clean reservoir 118 from the waste reservoir 120 which further militates against any potential mixture of the waste reservoir 120 with the clean reservoir 118. The nasal cleaning device 100 may include a removable cap 140 disposed on the head assembly 108, as shown in FIGS. 1-9. The removable cap 140 may cover an entirety of the head assembly 108. The removable cap 140 protects the head assembly 108 and the rotatable tip 110 when the nasal cleaning device 100 is not in use. Advantageously, the removable cap 140 also may include a compartment to store extra rotatable tips 110.

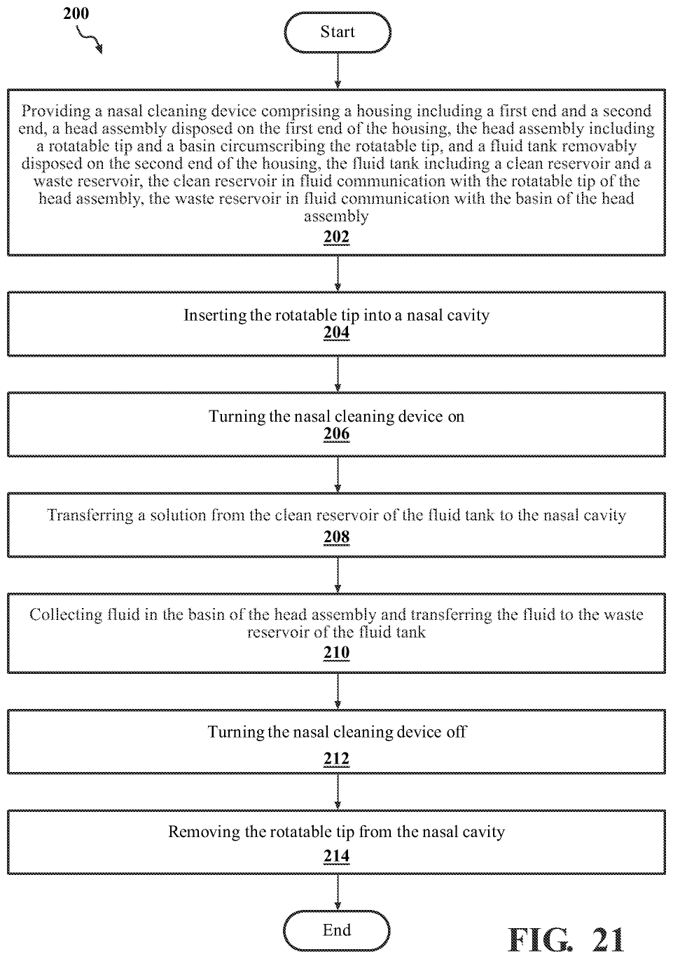

With reference now to FIG. 21, a flowchart representation of a method 200 of using a nasal cleaning device 100 is shown in accordance with an embodiment of the present disclosure. At 202, the method may include providing a nasal cleaning device 100, as described herein. At 204, the method may include inserting the rotatable tip 110 into a nasal cavity 101, as shown in FIG. 20. At 206, the method may include turning the nasal cleaning device 100 on. At 208, the method may include transferring a solution from the clean reservoir 118 of the fluid tank 116 to the nasal cavity 101. At 210, the method may include collecting fluid in the basin 112 of the head assembly 108 and transferring the fluid to the waste reservoir 120 of the fluid tank 116. At 212, the method may include turning the nasal cleaning device 100 off. At 214, the method may include removing the rotatable tip 110 from the nasal cavity 101.

Advantageously, the nasal cleaning device 100 provides an easy and efficient device to clean a nasal cavity 101. The rotatable tip 110 is easily removable and replaceable to allow a user to replace or clean the rotatable tip 110 after each use or to provide a rotatable tip 110 of a different size based on a preference of the user. The rotatable tip 110 may further oscillate +/−30 degrees relative to the vertical axis (A) to efficiently clean the nasal cavity 101. Advantageously, the basin 112 catches the liquid waste and solid waste released from the nasal cavity 101 to militate against any contact with the user. Desirably, transparency of the fluid tank 116 and a labeled fill line allows a user to determine when to refill the fluid tank 116 and to refill just enough to militate against overfill. Finally, the nasal cleaning device 100 is easy to use whereby a simple touch of a power button 150 turns the nasal cleaning device 100 on and drives the rotation of the rotatable tip 110 as well transfers the solution from the clean reservoir 118 to the rotatable tip 110.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods may be made within the scope of the present technology, with substantially similar results.

What is claimed is:

1. A nasal cleaning device comprising:
a housing including a first end and a second end;
a head assembly disposed on the first end of the housing, the head assembly including a rotatable tip and a basin circumscribing the rotatable tip; and
a fluid tank disposed on the second end of the housing, the fluid tank including a clean reservoir and a waste reservoir, the clean reservoir in fluid communication with the rotatable tip of the head assembly, the waste reservoir in fluid communication with the basin of the head assembly.

2. The nasal cleaning device of claim 1, wherein the rotatable tip of the head assembly is configured to rotate through at least an entire revolution.

3. The nasal cleaning device of claim 1, wherein the rotatable tip of the head assembly is configured to oscillate.

4. The nasal cleaning device of claim 3, further comprising a first motor configured to drive the rotatable tip of the head assembly.

5. The nasal cleaning device of claim 4, further comprising:
a first gear coupled to the first motor;
a second gear and a third gear directly coupled to a drive shaft, the drive shaft coupled to the rotatable tip of the head assembly;
wherein the first gear has an engagement portion and a nonengagement portion, rotation of the first gear resulting in the engagement portion engaging one of the second gear and the third gear and the nonengagement portion engaging the other of the second gear and the third gear, engagement of second gear causing the second gear to rotate in a first direction and engagement of the third gear causing the third gear to rotate in a second direction.

6. The nasal cleaning device of claim 3, wherein the rotatable tip is configured to oscillate +/−30 degrees about a vertical axis.

7. The nasal cleaning device of claim 1, wherein the rotatable tip of the head assembly is removably attached to the head assembly.

8. The nasal cleaning device of claim 1, wherein the rotatable tip is disposed entirely above a lip of the basin.

9. The nasal cleaning device of claim 1, wherein the rotatable tip of the head assembly includes a plurality of channels in fluid communication with the clean reservoir.

10. The nasal cleaning device of claim 1, wherein the basin of the head assembly includes an aperture disposed within the basin, the waste reservoir in fluid communication with the basin via the aperture.

11. The nasal cleaning device of claim 1, wherein the head assembly further includes a rotatable tip driver connected to the rotatable tip.

12. The nasal cleaning device of claim 11, wherein the rotatable tip driver is removably attached to the head assembly.

13. The nasal cleaning device of claim 1, wherein the clean reservoir is in fluid communication with the rotatable tip of the head assembly via a first tube, a second tube, a third tube, and a diaphragm pump, the diaphragm pump configured to provide fluid flow from the clean reservoir to the rotatable tip of the head assembly.

14. The nasal cleaning device of claim 1, wherein the waste reservoir is in fluid communication with the basin of the head assembly via a fourth tube, the fourth tube configured to permit a gravity flow from the basin of the head assembly to the waste reservoir.

15. The nasal cleaning device of claim 1, wherein the clean reservoir is separated from the waste reservoir by a wall.

16. The nasal cleaning device of claim 1, wherein the fluid tank further includes a drain disposed adjacent the waste reservoir, the drain configured to drain potential leakage into the waste reservoir.

17. The nasal cleaning device of claim 1, further including a removable cap disposed on the head assembly.

18. The nasal cleaning device of claim 17, wherein the removable cap covers an entirety of the head assembly.

19. The nasal cleaning device of claim 1, wherein:
a first motor is configured to drive the rotatable tip of the head assembly,
a first gear is coupled to the first motor,
a second gear and a third gear is directly coupled to a drive shaft, the drive shaft coupled to the rotatable tip of the head assembly, the first gear has an engagement portion and a nonengagement portion, rotation of the first gear resulting in the engagement portion engaging one of the second gear and the third gear and the nonengagement portion engaging the other of the second gear and the third gear, engagement of second gear causing the second gear to rotate in a first direction and engagement of the third gear causing the third gear to rotate in a second direction,
the rotatable tip of the head assembly is removably attached to the head assembly,
the rotatable tip is disposed entirely above a lip of the basin,
the rotatable tip of the head assembly includes a plurality of channels in fluid communication with the clean reservoir,
the basin of the head assembly includes an aperture disposed within the basin, the waste reservoir in fluid communication with the basin via the aperture,
the head assembly includes a rotatable tip driver connected to the rotatable tip,
the rotatable tip driver is removably attached to the head assembly,
the clean reservoir is in fluid communication with the rotatable tip of the head assembly via a first tube, a second tube, a third tube, and a diaphragm pump, the diaphragm pump configured to provide fluid flow from the clean reservoir to the rotatable tip of the head assembly,
the waste reservoir is in fluid communication with the basin of the head assembly via a fourth tube, the fourth tube configured to permit a gravity flow from the basin of the head assembly to the waste reservoir,
the clean reservoir is separated from the waste reservoir by a wall,
the fluid tank includes a drain disposed adjacent the waste reservoir, the drain configured to drain potential leakage into the waste reservoir, and
a removable cap is disposed on the head assembly, the removable cap configured to cover an entirety of the head assembly.

20. A method of using a nasal cleaning device, the method comprising:
providing a nasal cleaning device comprising a housing including a first end and a second end, a head assembly disposed on the first end of the housing, the head assembly including a rotatable tip and a basin circumscribing the rotatable tip, and a fluid tank disposed on the second end of the housing, the fluid tank including a clean reservoir and a waste reservoir, the clean reservoir in fluid communication with the rotatable tip of the head assembly, the waste reservoir in fluid communication with the basin of the head assembly;
inserting the rotatable tip into a nasal cavity;
turning the nasal cleaning device on;
transferring a solution from the clean reservoir of the fluid tank to the nasal cavity;
collecting fluid in the basin of the head assembly and transferring the fluid to the waste reservoir of the fluid tank;
turning the nasal cleaning device off; and
removing the rotatable tip from the nasal cavity.
